(19) United States
(12) Patent Application Publication
(10) Pub. No.: US 2016/0331499 A1
(43) Pub. Date: Nov. 17, 2016

(54) IMPROVED PLAQUE IDENTIFICATION INDEX

(71) Applicant: COLGATE-PALMOLIVE COMPANY, New York, NY (US)

(72) Inventors: Farid Ayad, Mississauga (CA); Salim A. Nathoo, Piscataway, NJ (US); Augusto Elias, San Juan (PE); Bernal Stewart, Somerset, NJ (US); Foti Panakagos, Cranford, NJ (US)

(73) Assignee: Colgate-Palmolive Company, New York, NY (US)

(21) Appl. No.: 15/106,281

(22) PCT Filed: Dec. 20, 2013
(86) PCT No.: PCT/US2013/076910

§ 371 Date: Jun. 18, 2016

(51) Int. Cl. A61C 19/04; A61B 5/00

(52) U.S. Cl. CPC A61C 19/04; A61B 5/4547

(57) ABSTRACT

Improvements in plaque removal have been achieved by the applicants' modifications to the Rustogi modification of the Modified Navy Plaque Index. The modification comprise of dividing a tooth into nine separate regions, visual examination each region for plaque; and scoring each region to obtain a plaque index number.

### BACKGROUND

Mechanical removal of plaque by toothbrushing is greatly dependent on the skills, perseverance, and motivation of the individual, and is therefore highly variable and inconsistent in the general population. Since supragingival plaque is a precursor of calculus, caries, and gingivitis, its daily removal by toothbrushing is necessary for decreasing these problematic sequences. Rustogi et al., J. Clin. Dent., vol. III, sppl. C: C9-C12 (1992).

One of the earlier indices for determining the amount of plaque on the tooth is the Modified Navy Plaque Index (MNPI) which is depicted in FIG. 1. Elliot et al., J. Periodontol., 43: 221-224 (1972).

An improvement known as the Rustogi modification of the Modified Navy Plaque Index (Rustogi MNPI) was developed nearly 20 years later and is depicted in FIG. 2. Rustogi et al., C10. The Rustogi MNPI differs from MNPI in that the former increases the tooth scoring areas in both the interproximal (mesial and distal) and gumline regions.

A mean plaque index (MPI) is calculated by summing the total number of tooth areas classified as having plaque present divided by the total number of tooth areas scored (e.g. 16 teeth classified as having plaque out of 32 teeth scored would have an MPI of 0.5). The efficacy of plaque removal can then be determined by comparing the MPI before and after the attempted plaque removal activity (e.g. brushing the teeth or rinsing the mouth).

However, despite the use of multiple regions of the tooth, one problem with the Rustogi MNPI is the “all or nothing” approach (1 for plaque, 0 for no plaque) which can mask significant changes in anti-plaque activity.

Despite these previously known improvements in plaque indices and in oral care compositions such a toothpastes, mouthwashes and mouthrinses and in oral care cleaning devices such as toothbrushes, conditions related to the accumulation of plaque such as gingivitis and periodontitis remain as oral health care problems. As such, a need for better identification of plaque on the tooth is still needed in the art.

### BRIEF SUMMARY

Surprisingly, improvements in plaque removal can be achieved by the applicants' modifications to the Rustogi modification of the Modified Navy Plaque Index.

The modification comprise of dividing a tooth into nine separate regions as depicted in FIG. 3, visual examination each region for plaque; and scoring each region to obtain a plaque index number.

In one embodiment of the invention, the effectiveness of a plaque removal technique, device or composition can be determined by measuring the difference in the plaque index number before and after application of the plaque removal technique, device or composition.

In another embodiment of the invention, an optimal plaque removal technique, device or composition is selected by determining the difference in plaque index number before and after application for a first plaque removal technique, device or composition and comparing against the difference in plaque index number before and after application for a second plaque removal technique, device or composition wherein the technique, device or composition with the greatest difference in plaque index number is selected.

In another embodiment of the invention is the treatment of gingivitis or periodontitis which comprises using the optimal plaque removal technique or administering the optimal device or composition to the oral cavity of a patient in need thereof.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

FIG. 1 depicts a diagram of the Modified Navy Plaque Index.

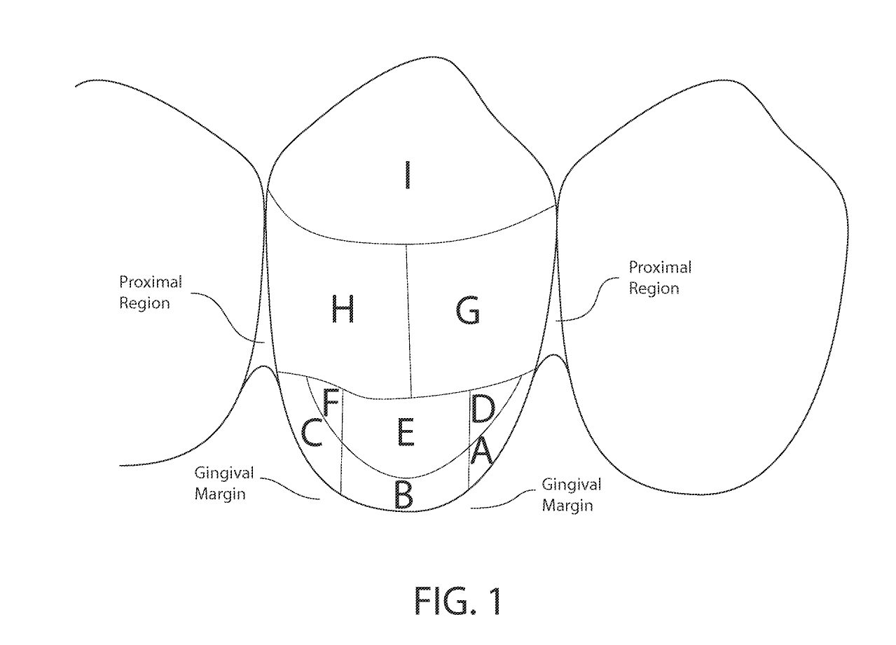

FIG. 2 depicts a diagram of the Rustogi modification of the Modified Navy Plaque Index.

FIG. 3 depicts a diagram of the applicants' modification Rustogi modification of the Modified Navy Plaque Index of the present invention.

### DETAILED DESCRIPTION

The following description of the preferred embodiment(s) is merely exemplary in nature and is in no way intended to limit the invention, its application, or uses.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

For the purposes of this invention, the term “tooth” is intended to encompass not only a naturally occurring tooth in a mammal (e.g. humans), but also artificial forms thereof (e.g. dentures, hydroxyapatite discs).

For the purposes of this invention, the phrase “visual examination” refers to determination of plaque without the use of electronics or instruments to aid in the determination. However, visual examination can include digital superimposition of the applicants' modification of the MNPI on the tooth. Visual examination can be made directly by the observer or indirectly, e.g. taking a photograph of the patient's teeth for ex vivo evaluation of the plaque on the teeth.

In one embodiment of the invention, the improved method for the visual determination of plaque on a tooth which comprises:

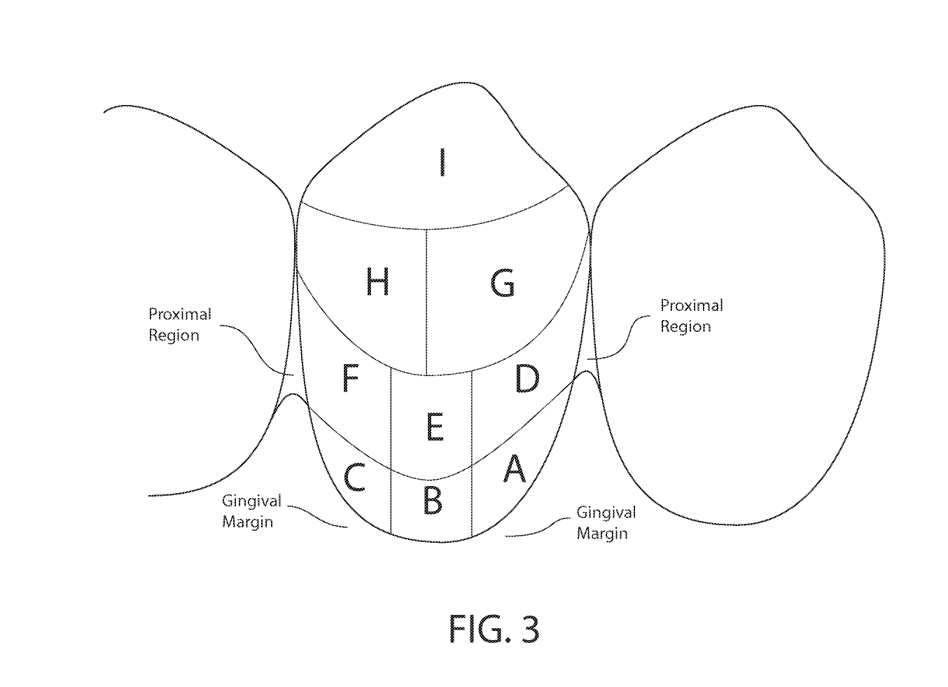

a. dividing a tooth into three to nine separate regions A-I as depicted in FIG. 3,

b. visual examination each region for plaque;

c. scoring each region as:
i. 0, if no plaque;
ii. 1. if plaque covers less than one-third of the surface of the tooth;
iii. 2, if plaque covers less than two-thirds of the surface of the tooth;
iv. 3, if plaque cover two-thirds or more of the surface of the tooth; and

d. totaling the scores for each tooth examined to obtain a plaque index number.

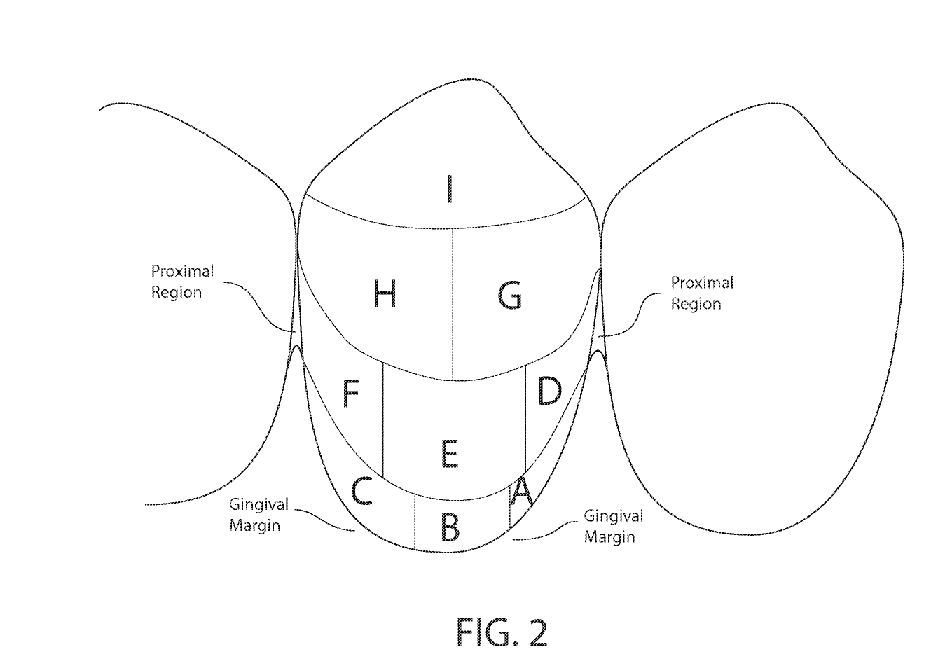

The curves depicted in FIG. 3 are substantially the same as the curves from the Rustogi modification (see FIG. 2) with the exception that the vertical lines in separating region F and E and separating region E and D form a straight line with the vertical line separating region C and B and separating region B and A.

The lower boundary of regions A, B and C can be defined by the gum line and the top boundary does not rise above the papillae (the top point of the gums between the teeth); this top boundary of regions A, B and C constitutes the lower boundary of regions D, E, and F.

The top boundary of D, E, and F is defined by the lower contact point of a tooth with an adjacent tooth; this top boundary of regions D, E, and F is the lower boundary for regions G and H.

The top boundary of regions G and F is the uppermost contact point of a tooth with an adjacent tooth; this top boundary of regions G and F constitutes the lower boundary of region I.

The top boundary of region I extend to the uppermost boundary of the tooth.

The tooth depicted in FIG. 3 is a canine tooth, but is intended to be representative of all tooth types, i.e. incisors, canines, pre-molars and molars and also representative of both the set of teeth in the lower and upper jaw.

In another embodiment of the invention, the improved method for the visual determination of plaque on a tooth which comprises:

a. dividing a tooth into three to nine separate regions as depicted in FIG. 3,

b. visual examination each region for plaque; and

c. scoring each region as:
i. 0, if no plaque;
ii. 1, if separate flecks of plaque covers less than one-third of the surface of the tooth;
iii. 2, if a continuous band of plaque covers less than one-third of the surface of the tooth;
iv. 3. if plaque covers less than two-thirds of the surface of the tooth;
v. 4, if plaque cover two-thirds or more of the surface of the tooth; and

d. totaling the scores for each tooth examined to obtain a plaque index number.

In another embodiment of the invention, in the improved method, the tooth is divided into three different regions selected from the group consisting of: (1) regions A, B and C; (2) regions D, E and F; and (3) regions G, H, and I (each of the letter referring to the region defined in FIG. 3)

In another embodiment of the invention, in the improved method, the tooth is divided into six different regions selected from the group consisting of: (1) regions A, B, C, D, E, and F; (2) regions D, E, F, G, H and I; and (3) regions A, B, C, G, H, and I (each of the letter referring to the region defined in FIG. 3)

In another embodiment of the invention, in the improved method, the tooth is divided into nine different regions consisting of A, B, C, D, E, F, G, H and I (each of the letter referring to the region defined in FIG. 3).

In another embodiment of the invention, each method of visually determining plaque on a tooth optionally includes a step of staining the plaque prior to visual determination. The staining can be accomplished via any orally acceptable staining material which binds to plaque. These staining materials includes but is not limited to any safe and approved plaque disclosing dyeing solution or tablet, e.g. those solutions or tablets containing erythrosine, fluorescein dye or a food coloring.

In one embodiment of the invention, the effectiveness of a plaque removal technique, device or composition can be determined by measuring the difference in the plaque index number before and after application of the plaque removal technique, device or composition.

In one embodiment of the invention, the plaque removal technique includes, but is not limited to brushing, flossing and rinsing; the device includes, but is not limited to a toothbrush (which includes electric toothbrushes), floss, dental strip, film, syringe, tape, gum, pill, and liquid sprayers; and the composition includes, but is not limited to a dentifrice, e.g., a toothpaste, dental gel, dental cream, or tooth powder, a mouthwash, mouth rinse, or mouth spray; an oral slurry or liquid dentifrice; a gum or other confectionary; a lozenge; dental floss or dental tape; a prophylaxis paste or powder; a mono- or multi-layer oral film or gel strip, e.g., tooth strips or breath strips, preferably using a biodegradable or orally consumable film or gel; functional film or gel flakes or functional milli-, micro-, or nano-particles; a film-forming composition comprising pre-gel(s) or pre-polymer(s), e.g., film-forming dentifrices, dental paints; a tooth hardener; or a coating on an oral, e.g., orthodontic, appliance or implant.

In another embodiment of the invention, the composition comprises of an anti-plaque agent which includes, but is not limited to stannous, copper, magnesium and strontium salts, dimethicone copolyols such as cetyl dimethicone copolyol, papain, glucoamylase, glucose oxidase, urea, calcium lactate, calcium glycerophosphate, strontium polyacrylates and mixtures thereof.

In another embodiment of the invention, the composition comprises of a tartar control (anticalculus) agent. Tartar control agents among those useful herein include phosphates and polyphosphates (for example pyrophosphates), polyaminopropanesulfonic acid (AMPS), polyolefin sulfonates, polyolefin phosphates, diphosphonates such as azacycloalkane-2,2-diphosphonates (e.g., azacycloheptane-2,2-diphosphonic acid), N-methyl azacyclopentane-2,3-diphosphonic acid, ethane-1-hydroxy-1,1-diphosphonic acid (EHDP) and ethane-1-amino-1,1-diphosphonate, phosphonoalkane carboxylic acids and salts of any of these agents, for example their alkali metal and ammonium salts. Useful inorganic phosphate and polyphosphate salts include monobasic, dibasic and tribasic sodium phosphates, sodium tripolyphosphate, tetrapolyphosphate, mono-, di-, tri- and tetrasodium pyrophosphates, sodium trimetaphosphate, sodium hexametaphosphate and mixtures thereof, wherein sodium can optionally be replaced by potassium or ammonium. Other useful anticalculus agents include polycarboxylate polymers and polyvinyl methyl ether/maleic anhydride (PVME/MA) copolymers, such as those available under the Gantrez™ brand from ISP, Wayne, N.J.

In another embodiment of the invention, an optimal plaque removal technique, device or composition is selected by determining the difference in plaque index number before and after application for a first plaque removal technique, device or composition and comparing against the difference in plaque index number before and after application for a second plaque removal technique, device or composition wherein the technique, device or composition with the greatest difference in plaque index number is selected.

In another embodiment of selecting the optimal plaque removal technique, device or composition, the selected technique, device or composition provides at 20%-80% greater plaque reduction in the whole mouth (all teeth) when tested using the applicants modification of the Rustogi modification of the Navy Plaque Index of the invention instead of the Rustogi MNPI.

In another embodiment of this invention, the selected technique, device or composition provides at 30%-50% greater plaque reduction in the whole mouth (all teeth) when tested using the applicants' modification of the Rustogi modification of the Navy Plaque Index of the invention instead of the Rustogi MNPI.

In another embodiment of selecting the optimal plaque removal technique, device or composition, the selected technique, device or composition provides at 20%-80% greater plaque reduction in the gumline (regions A-C) when tested using the applicants' modification of the Rustogi modification of the Navy Plaque Index of the invention instead of the Rustogi MNPI. In another embodiment of this invention, the selected technique, device or composition provides at 30%-50% greater plaque reduction in the gumline (regions A-C) when tested using the applicants' modification of the Rustogi modification of the Navy Plaque Index of the invention instead of the Rustogi MNPI.

In another embodiment of the invention, determination of the optimal plaque removal is achieved by comparing like agents (e.g. first toothbrush vs. second toothbrush) or different agents (e.g. toothbrush vs. toothpaste).

Another embodiment of the invention is directed toward the treatment of gingivitis or periodontitis which comprises using the optimal plaque removal technique or administering the optimal device or composition to the oral cavity of a patient in need thereof.

In another embodiment of the treatment of gingivitis or periodontitis, the patient in need thereof does not have dental caries.

Another embodiment of the invention is directed toward a method of determining plaque accumulation and the effectiveness of a plaque removal technique which comprises of prophying the teeth with an oral care composition or using an oral care device and evaluating the plaque accumulation after a pre-determined period of time with the plaque index of the invention.

In another embodiment of the method of determining plaque accumulation and the effectiveness of a plaque removal technique, the pre-determined time can be a time range selected from the group consisting of 1 hour to 24 weeks, 6-12 hours, 8-16 hours, 24-48 hours, 1-2 weeks, 4-8 weeks and 1-24 weeks.

In another embodiment of the method of determining plaque accumulation and the effectiveness of a plaque removal technique, multiple evaluations of plaque accumulation within a pre-determined time are made after a single prophying of the teeth with an oral care composition or use of an oral care device.

In another embodiment of the method of determining plaque accumulation and the effectiveness of a plaque removal technique, multiple evaluations of plaque accumulation within a pre-determined time are made after a single prophying of the teeth with an oral care composition or use of an oral care device.

In another embodiment of the method of determining plaque accumulation and the effectiveness of a plaque removal technique, multiple evaluations of plaque accumulation within a pre-determined time are made after a each prophying of the teeth with an oral care composition or use of an oral care device.

In each of the embodiment of the invention described above, the method of visually determining plaque occurs in vivo or ex vivo. In one embodiment of ex vivo plaque determination, the determination occurs via photographic or electronic representation of the teeth. In another embodiment of ex vivo plaque determination, the teeth themselves are removed from the body for visual determination of plaque (e.g. removal of dentures).

Embodiments of the present invention are further described in the following examples. The examples are merely illustrative and do not in any way limit the scope of the invention as described and claimed.

### EXAMPLES

### Example 1

Test subjects refrained from any oral hygiene procedures for at 24 hours prior to their examination and not eating, drinking and smoking for four hours prior to their examination. All subjects received an evaluation of their oral soft tissues by one or more clinical examiners which included an evaluation of the soft and hard palate, gingival mucosa, buccal mucosa, mucogingival fold areas, tongue, sublingual and submandibular areas, salivary glands and the tonsilar and pharyngeal areas.

All subjects are provided with a soft-bristled adult toothbrush and a tube of commercially available fluoridated toothpaste for home use. They will be instructed to brush their teeth twice daily (morning and evening) for one minute.

After approximately 7-10 days of product use, the subjects reported to the clinical facility having refrained from any oral hygiene procedures for at 24 hours prior to their examination and after not eating, drinking and smoking for four hours prior to their examination. They receive an evaluation of their oral soft tissues.

After soft tissue evaluation, subjects will be evaluated by one or more clinical examiners using either the Rustogi MNPI or the new methodology of the invention (i.e. applicants' modification of the Rustogi modification of the modified Navy Plaque Index) with the results being recorded by the clinical examiner.

Assessment of the oral soft and hard tissues was conducted via a visual examination of the oral cavity and peri-oral area using a dental light and dental mirror. Examination of the gingiva (free and attached), labial mucosa, oropharynx/mucobuccal folds, lips, and perioral area was completed at all dental examination visits.

The scoring under the Rustogi modification only distinguishes between whether there is or there is not plaque present in each of the 9 selected regions. Evaluation of one patient's plaque score under the Rustogi modification revealed a score of 9 before and after brushing the teeth.

However, scoring the same 9 selected regions for the same patient with the applicants' modification of the Rustogi modification of the modified Navy Plaque Index) of the invention revealed that their plaque score was nearly 26% lower (starting score of 27; after brushing score of 20), i.e. showed the true efficacy of the toothbrush.

Tables 1 and 2 show the Rustogi MNPI plaque scoring vs. the scoring based in the applicants' modification of the Rustogi modification of the modified Navy Plaque Index of the invention for the nine subjects who underwent clinical testing with the 360™ Toothbrush (Colgate-Palmolive). Data from both table represents scoring from the whole mouth.

TABLE 1
(Rustogi MNPI scoring)

| Toothbrush | Baseline | 7 day | Within-treatment |
|---|---|---|---|
| type | summary | summary | analysis (% change) |
| 360 ™ | 0.54 | 0.58 | 7.4 |

TABLE 2
(scoring of the applicants' modification of the
Rustogi modification of the modified Navy Plaque Index)

| Toothbrush | Baseline | 7 day | Within-treatment |
|---|---|---|---|
| type | summary | summary | analysis (% change) |
| 360 ™ | 1.17 | 0.92 | −21.3 |

Comparing the data between Table 1 and Table 2, it can be seen that the Rustogi MNPI scoring method was less effective in determining the overall effectiveness of the 360™ toothbrush than the applicants' modification of the Rustogi modification of the modified Navy Plaque Index, i.e. an observer using Rustogi MNPI would have thought the amount of plaque increased by 7.4% when in actuality, the amount of plaque decreased by 21.3%.

The scored data of the present method is consistent with the examining clinician of the studies that after 7 days of brushing with the 360™ toothbrush, there were less visible plaques detected on the test subjects' teeth.

The whole mouth data from Tables 1 and 2 also hold forth when focusing on specific section of the tooth, e.g. regions A. B and C (gumline region) and regions D and F (interproximal region). See Table 3 below.

TABLE 3
(Difference in plaque reduction in various regions of the tooth)

| Scoring method | Whole mouth | Gumline | Interproximal |
|---|---|---|---|
| applicants' modification of | 27.6 | 33.0 | 25.0 |
| the Rustogi MNPI | | | |
| Rustogi MNPI | −3.6 | 1.5 | 10.2 |

As can be seen from the data in Table 3, the preference for 360™ toothbrush is especially enhanced for use in plaque removal at the gumline and as such is well situated for treating or preventing gingivitis or periodontitis.

Those of skill in the art will appreciate that use of the applicants' modification of the Rustogi modification of the Navy Plaque Index of the invention can extend to other oral care techniques, device and compositions and serves as a valuable tool for the practitioner to select the optimal for reducing plaque. Furthermore, selecting the optimal agent for reducing plaque also facilitates the treatment or prevention of gingivitis or periodontitis.

As those skilled in the art will also appreciate, numerous changes and modifications may be made to the embodiments described herein without departing from the spirit of the invention. It is intended that all such variations fall within the scope of the appended claims.

1. An improved method for the visual determination of plaque on a tooth which comprises:
a. dividing a tooth into nine separate regions A-I as depicted in FIG. 3,
b. visual examination each region for plaque; and
c1. scoring each region as:
i. 0, if no plaque;
ii. 1, if plaque covers less than one-third of the surface of the tooth;
iii. 2, if plaque covers less than two-thirds of the surface of the tooth;
iv. 3, if plaque cover two-thirds or more of the surface of the tooth; or
c2. scoring each region as:
i. 0, if no plaque;
ii. 1, if separate flecks of plaque covers less than one-third of the surface of the tooth;
iii. 2, if a continuous band of plaque covers less than one-third of the surface of the tooth;
iv. 3, if plaque covers less than two-thirds of the surface of the tooth;
v. 4, if plaque cover two-thirds or more of the surface of the tooth;
d. totaling the scores for each tooth examined to obtain a plaque index number.

2. The improved method of claim 1, wherein steps a.-c. are repeated for one or more tooth.

3. The improved method of claim 1, wherein the visual determination is made in vivo.

4. The improved method of any claim 1, wherein the visual determination is made ex vivo.

5. The improved method of claim 1 wherein the tooth is stained prior to visual examination.

6. A method for quantitating the efficacy of a plaque removal technique, device or composition in the removal of plaque from a tooth, the method comprising:
(a1) quantitatively determining the amount of plaque on the tooth by the method of claim 1,
(b1) treating the tooth with a plaque removal technique, device or composition;
(c1) quantitatively determining the amount of plaque on the tooth by the method of claim 1 after (b1), wherein the difference between (c1) and (a1) indicates the efficacy of said oral care product in plaque removal.

7. The method of claim 6, wherein steps (a1)-(c1) are repeated for another plaque removal technique, device or composition and an optimal plaque removal technique, device or composition is selected by determining the difference in plaque index number before and after application for a first plaque removal technique, device or composition and comparing against the difference in plaque index number before and after application for a second plaque removal technique, device or composition wherein the technique, device or composition with the greatest difference in plaque index number is selected.

8. The method of claim 7, wherein the selected technique, device or composition provides at least 20%-80% greater plaque reduction in the whole mouth (all teeth).

9. The method of claim 7, wherein the selected technique, device or composition provides at least 20%-80% greater plaque reduction in the gumline.

10. The method of claim 6, wherein the optimal plaque removal technique, device or composition is used in a step (dl) to treat or prevent gingivitis or periodontitis by administering the plaque removal technique, device or composition to a patient in need thereof.

11. The method of claim 6, wherein the plaque removal technique is selected from the group consisting of brushing, flossing and rinsing; the device is selected from the group consisting of a toothbrush (which includes electric toothbrushes), floss, syringe, and liquid sprayers; and the composition is selected from the group consisting of toothpaste, dental gel, dental cream, or tooth powder; a mouthwash, mouth rinse, or mouth spray; an oral slurry or liquid dentifrice; a gum or other confectionary; a lozenge; dental floss or dental tape; a prophylaxis paste or powder; a mono- or multi-layer oral film or gel strip, tooth strips or breath strips, functional film or gel flakes or functional milli-, micro-, or nano-particles; a film-forming composition comprising pre-gel(s) or pre-polymer(s), and a dental paint.

12. The method of claim 6, wherein the dentifrice is a toothbrush, toothpaste, mouthwash, or mouthrinse.

13. The method of claim 6, wherein steps (a1)-(c1) are repeated for one or more tooth.

14. The method of claim 6, wherein the visual determination is achieved in vivo.

15. The method of claim 6 wherein the visual determination is achieved ex vivo.

16. The method of claim 6, wherein the tooth is stained prior to visual examination.
